(19) United States
(12) Patent Application Publication
(10) Pub. No.: US 2025/0205431 A1
(43) Pub. Date: Jun. 26, 2025

(54) SYRINGE BASE, SYRINGE SUPPORT, AND SYRINGE ASSEMBLY

(71) Applicants: JANMAN PRECISION INDUSTRY CO., LTD., Taoyuan City (TW); TRANSIN BIOMEDICAL CO., LTD., Taoyuan City (TW)

(72) Inventors: CHIEH-MAO LIN, Taoyuan City (TW); TZU-HSUAN HUANG, Taoyuan City (TW); YI-JHE WU, Taoyuan City (TW)

(73) Assignees: JANMAN PRECISION INDUSTRY CO., LTD., Taoyuan City (TW); TRANSIN BIOMEDICAL CO., LTD., Taoyuan City (TW)

(21) Appl. No.: 18/732,660

(22) Filed: Jun. 4, 2024

(30) Foreign Application Priority Data
Dec. 25, 2023 (TW) 112150581

(51) Int. Cl. A61M 5/31

(52) U.S. Cl. CPC A61M 5/3137

(57) ABSTRACT

A syringe base includes a base body, an abutting component and at least one engaging component. The base body includes a position-limiting portion and has a position-limiting chamber and a support channel. The position-limiting chamber adjoins the position-limiting portion. The support channel is in communication with the position-limiting chamber and penetrates the base body. The abutting component is disposed at an end of the base body and has a base opening and a position-limiting component slot. The base opening is in communication with the support channel. The position-limiting component slot is disposed outside the base opening and adjoins the position-limiting chamber. The engaging component is disposed at the abutting component, protrudes relative to the abutting component and is adapted to bend or rotate relative to the abutting component. A syringe support and a syringe assembly are further provided.

### CROSS-REFERENCE TO RELATED APPLICATION

This non-provisional application claims priority under 35 U.S.C. § 119(a) on patent application Ser. No. 11/215,0581 filed in Taiwan, R.O.C. on Dec. 25, 2023, the entire contents of which are hereby incorporated by reference.

### BACKGROUND OF THE INVENTION

### 1. Field of the Invention

The present disclosure relates to a syringe base, a syringe support, and a syringe assembly, and in particular to a syringe base, a syringe support, and a syringe assembly for containing a syringe and concealing the needle of the syringe after the syringe has run out of its contents.

### 2. Description of the Related Art

People currently attach great importance to therapeutic safety. In general, to administer medication to a person by injection or perform a blood test on the person regardless of whether the person is sick, injured, or at an advanced age, medical professionals use a syringe to give fluids, medicine, nutrition, or blood directly into the person's blood stream or draw blood from the person's body. However, when a needle of the used syringe is exposed, not only is the needle likely to injure the medical professionals, but blood-borne infections may also occur and thus spread severe diseases.

### BRIEF SUMMARY OF THE INVENTION

It is an objective of the present disclosure to provide a syringe base, a syringe support, and a syringe assembly for containing a syringe and concealing the needle of the syringe after the syringe has run out of its contents so as to enhance ease of use and safety for medical professionals.

To achieve the above and other objectives, the present disclosure provides a syringe base including a base body, an abutting component and at least one engaging component. The base body includes a position-limiting portion and has a position-limiting chamber and a support channel. The position-limiting chamber adjoins the position-limiting portion and extends in a feeding direction. The support channel is in communication with the position-limiting chamber and extends in the feeding direction to penetrate the base body. The abutting component is disposed at an end of the base body in the feeding direction and has a base opening and a position-limiting component slot. The base opening is in communication with the support channel. The position-limiting component slot is disposed outside the base opening in a radial direction of the base body and adjoins the position-limiting chamber. The engaging component is disposed at the abutting component, protrudes relative to the abutting component, and is adapted to bend or rotate relative to the abutting component.

In an embodiment, a width of a portion of the position-limiting component slot positioned distal to the position-limiting chamber in the radial direction is greater than a width of another portion of the position-limiting component slot positioned proximate to the position-limiting chamber in the radial direction.

In an embodiment, the syringe base further includes a guiding portion disposed at the base body and has a duct. The duct extends in the feeding direction and is in communication with the support channel.

In an embodiment, the abutting component further has a guiding component slot disposed outside the base opening in the radial direction. The duct is corresponding in position to a portion of the guiding component slot in the feeding direction.

In an embodiment, a width of the portion of the guiding component slot corresponding in position to the duct in the radial direction is smaller than a width of another portion of the guiding component slot without corresponding in position to the duct in the radial direction.

In an embodiment, the base body further has an engaging hole, and the engaging hole penetrates a portion of the guiding portion.

In an embodiment, the position-limiting portion and the guiding portion are spaced apart in a circumferential direction of the base body.

In an embodiment, the syringe base further includes a gripping portion disposed at the abutting component and adjoins the base opening.

In an embodiment, the engaging component is provided in a plurality. Each of the engaging components includes an engaging component sliding connection portion. A distance between each of the engaging component sliding connection portions and a central axis of the support channel in the radial direction gradually changes in the feeding direction.

In an embodiment, the engaging component includes two engaging component sliding connection portions. The syringe base is adapted to be assembled with a syringe support, and a gap between the engaging component sliding connection portions equals a wing component of the syringe support in size substantially.

In an embodiment, the syringe base is adapted to be assembled with a syringe. The syringe includes an operating portion. A distance between a circumference of the operating portion and a central axis of the support channel in the radial direction is greater than a distance between the engaging component and the central axis in the radial direction.

To achieve the above and other objectives, the disclosure further provides a syringe support including a support body, a position-limiting component, a carrying component, a guiding component and at least one wing component. The support body has a syringe channel. The syringe channel extends in a feeding direction and penetrates the support body. The position-limiting component is disposed at the support body and protrudes relative to a sidewall of the support body. The carrying component is disposed at the support body and has a support opening. The support opening is in communication with the syringe channel. The position-limiting component and the carrying component are disposed at a front end and a rear end of the support body in the feeding direction respectively. The guiding component is disposed at the support body, protrudes relative to the sidewall of the support body, and is spaced apart from the position-limiting component in a circumferential direction of the support body. The wing component is disposed at the carrying component, protrudes relative to the carrying component, and is adapted to bend or rotate relative to the carrying component.

In an embodiment, an end portion of the guiding component and the sidewall of the support body are separate from each other.

In an embodiment, the syringe support further includes two fastening components disposed at a front end and a rear end of the support body in the feeding direction respectively. A distance between a surface of the fastening components and a central axis of the syringe channel in a radial direction of the support body substantially equals an inner diameter of the syringe channel.

In an embodiment, the support body includes a resilient component abutting portion disposed at a front end of the support body in the feeding direction and adapted to abut against a resilient component.

In an embodiment, the support body further has a syringe observation slot extending in the feeding direction and penetrating the sidewall of the support body.

In an embodiment, The wing component is provided in a plurality. Each of the wing components includes a wing component sliding connection portion. A distance between each of the wing component sliding connection portions and a central axis of the syringe channel in a radial direction of the support body gradually changes in the feeding direction.

In an embodiment, the wing component is provided in a plurality, and the wing components are equally spaced apart in the circumferential direction.

In an embodiment, the syringe support is adapted to be assembled with a syringe including a body portion, and a distance between a circumference of the body portion and a central axis of the syringe channel in a radial direction of the support base is greater than a distance between the wing component and the central axis in the radial direction.

To achieve the above and other objectives, the present disclosure further provides a syringe assembly adapted to be assembled with a syringe. The syringe assembly includes a syringe base, a syringe support and a resilient component. The syringe support is disposed in the support channel. The syringe channel is adapted to receive the syringe. The resilient component is disposed between the syringe base and the syringe support.

Thus, according to the disclosure, the syringe base and the syringe support of the syringe assembly receive the syringe support and the syringe respectively. With the position-limiting component, position-limiting component slot and position-limiting chamber getting fixed in place consecutively, separation of the syringe base and the syringe support is impossible upon completion of the assembly process. Therefore, not only can a used syringe be contained in the syringe base, but the needle portion of the syringe can also be concealed, so as to enhance ease of use and safety of medical professionals.

The disclosure is hereunder illustrated by embodiments, depicted by accompanying drawings and described in detail to render the features and advantages of the disclosure clear and comprehensible.

### BRIEF DESCRIPTION OF THE DRAWINGS

FIG. 1 is a perspective view of a syringe assembly disclosed in an embodiment of the present disclosure and assembled with a syringe.

FIG. 2 is an exploded view based on FIG. 1.

FIG. 3 is a front view of a syringe base of FIG. 1.

FIG. 4 is a top view of the syringe base of FIG. 1.

FIG. 5 is a cross-sectional view taken along line X-X of FIG. 4.

FIG. 6 is a front view of a syringe support of FIG. 1.

FIG. 7 is a top view of the syringe support of FIG. 1.

FIG. 8 is a cross-sectional view taken along line Y-Y of FIG. 7.

FIG. 9 is a front view of the syringe support and the syringe shown in FIG. 1 and assembled together.

FIG. 10 is a perspective view of how a syringe support assembled with a syringe is assembled with a syringe base.

FIG. 11 is a top view of FIG. 10.

FIG. 12 is a perspective view of how the syringe support of FIG. 10 rotates relative to the syringe base.

FIG. 13 is a front view of the syringe support and the syringe base shown in FIG. 12 after assembled.

FIG. 14 is a front view of the syringe support and the syringe base shown in FIG. 13 after the syringe has run out of its contents.

FIG. 15 is a front view of how the syringe support of FIG. 14 moves relative to the syringe base.

FIG. 16 is a perspective view of a syringe assembly disclosed in an embodiment of the present disclosure and assembled with a syringe.

FIG. 17 is a perspective view of the syringe support of the syringe assembly according to an embodiment of the present disclosure.

FIG. 18 is a front view of FIG. 17.

### DETAILED DESCRIPTION OF THE INVENTION

The aforesaid and other technical contents, features and advantages of the disclosure are clearly presented through the description of the preferred embodiments illustrated by the accompanying drawings. Direction-related terms used in the embodiments below, such as “upper”, “lower”, “left”, “right”, “front” and “rear”, are intended to refer to the directions depicted in accompanying drawings. The direction-related terms are aimed at assisting with describing and understanding the embodiments of the disclosure rather than limiting the disclosure. Identical or similar components in the embodiments below are denoted by identical or similar reference numerals.

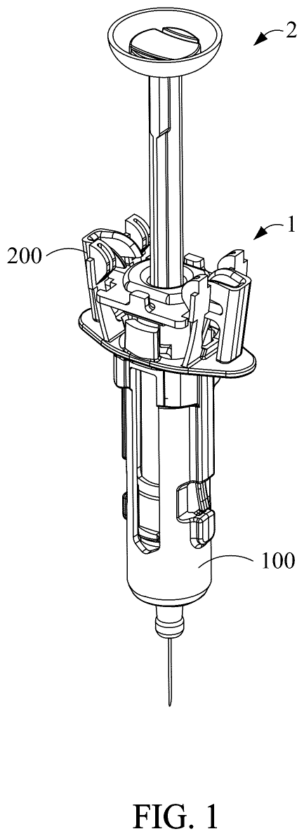

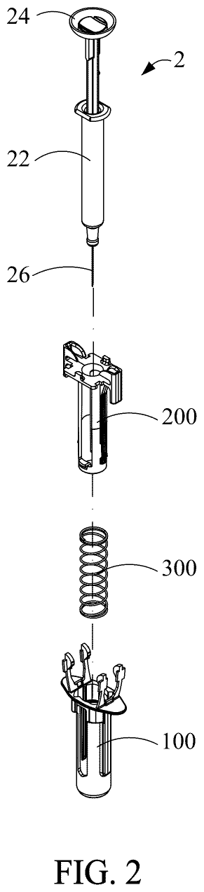

Refer to FIG. 1 and FIG. 2. FIG. 1 is a perspective view of a syringe assembly 1 disclosed in an embodiment of the disclosure and assembled with a syringe. FIG. 2 is an exploded view based on FIG. 1. In this embodiment, the syringe assembly 1 is adapted to coordinate with a syringe 2. The syringe 2 includes a body portion 22, an operating portion 24 and a needle portion 26. The body portion 22 contains a liquid medication or a biospecimen collected from a patient's body. The operating portion 24 is, for example, a head of a piston rod capable of pushing and moving the contents of the body portion 22 and is movably connected to the body portion 22. The needle portion 26 is, for example, adapted to pierce a patient's skin and is assembled with the body portion 22. The syringe assembly 1 includes a syringe base 100, a syringe support 200 and a resilient component 300. The syringe support 200 is disposed in the syringe base 100 and receives a portion of the syringe 2. The resilient component 300 is a linear spring and is disposed between the syringe base 100 and the syringe support 200.

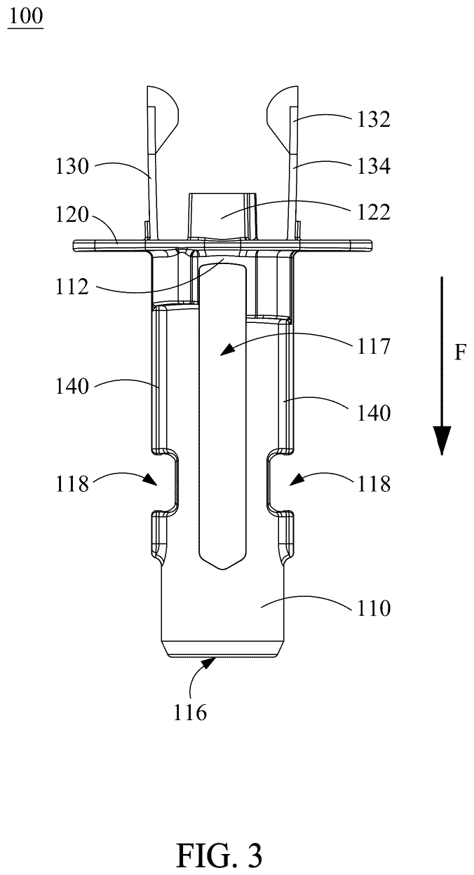

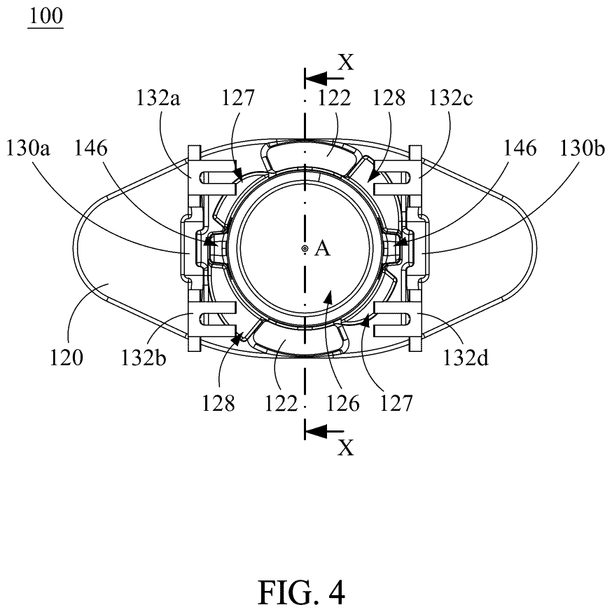

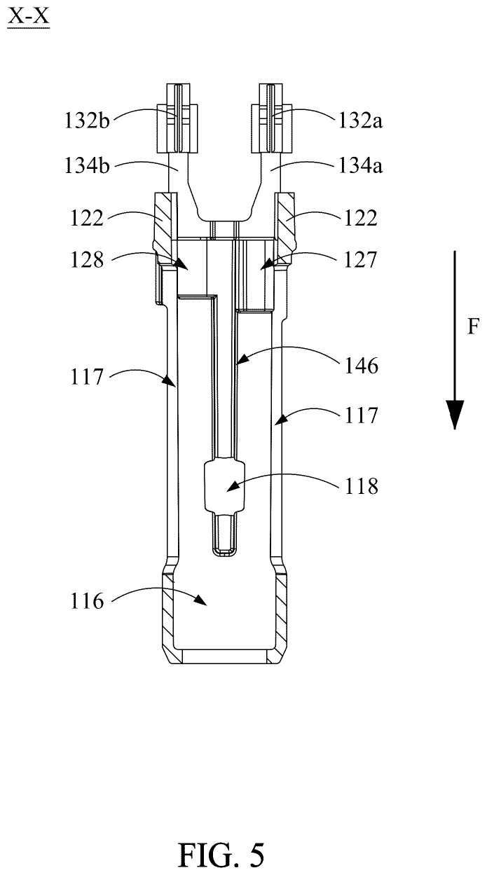

Refer to FIG. 3 through FIG. 5. FIG. 3 is a front view of a syringe base of FIG. 1. FIG. 4 is a top view of the syringe base of FIG. 1. FIG. 5 is a cross-sectional view taken along line X-X of FIG. 4. The syringe base 100 is a substantially hollow tubular structure and includes a base body 110, an abutting component 120 and at least one engaging component 130. The base body 110 includes a position-limiting portion 112 and has a support channel 116 and a position-limiting chamber 117. The support channel 116 extends in a feeding direction F and penetrates the base body 110. The position-limiting chamber 117 is in communication with the support channel 116, adjoins the position-limiting portion 112, and extends in the feeding direction F. It is noteworthy that in this embodiment the base body 110 has two position-limiting chambers 117. The two position-limiting chambers 117 overlap in the front view of FIG. 3 and are disposed on opposing sides of the base body 110 in a circumferential direction respectively as shown in FIG. 5.

The abutting component 120 is disposed at one end of the base body 110 in the feeding direction F and has a base opening 126. The base opening 126 is in communication with the support channel 116. This embodiment is exemplified by the abutting component 120 being disposed at the rear end of the base body 110 in the feeding direction F, but the disclosure is not limited thereto. The engaging component 130 is disposed at the abutting component 120 and protrudes relative to the abutting component 120. The engaging component 130 is, for example, in the number of two, and the two engaging components 130 are arranged symmetrically relative to the abutting component 120. For the sake of illustration, the engaging component 130 shown on the left in FIG. 4 is hereinafter referred to as the first engaging component 130a, and the engaging component 130 shown on the right in FIG. 4 is hereinafter referred to as the second engaging component 130b. The first engaging component 130a and the second engaging component 130b are adapted to bend sideward or rotate pivotally relative to the abutting component 120 respectively. Therefore, when the syringe support 200 is disposed in the syringe base 100, the abutting component 120 and the engaging component 130 of the syringe base 100 limit the positions of the front, end and the rear end of the syringe support 200 in the feeding direction F respectively, allowing the syringe support 200 to be gripped and fastened in place at a specific point of the syringe base 100.

As shown in FIG. 4 and FIG. 5, the abutting component 120 further has a position-limiting component slot 127. The position-limiting component slot 127 is disposed outside the base opening 126 in a radial direction of the base body 110 and adjoins the position-limiting chambers 117. In this embodiment, the position-limiting component slot 127 is, for example, in the number of two, and the two position-limiting component slots 127 correspond in position to the position-limiting chambers 117 on the left and on the right in FIG. 5 respectively. In some feasible embodiments, the width of the position-limiting component slot 127 gradually changes in the circumferential direction of the base body 110 such that the width of the position-limiting component slot 127 gradually decreases to preclude variation in any physical dimension in the radial direction at the junction of the position-limiting component slot 127 and each position-limiting chamber 117 whenever the position-limiting component slot 127 gradually approaches the position-limiting chambers 117 along the circumferential direction. Thus, a portion of the position-limiting component slot 127 is positioned distal to the position-limiting chamber 117 and has in the radial direction a width greater than a width, in the radial direction, of another portion of the position-limiting component slot 127 when the another portion of the position-limiting component slot 127 is positioned proximate to the position-limiting chamber 117. Therefore, the syringe support 200 and the syringe base 100 can be assembled with each other smoothly.

The syringe base 100 further includes a guiding portion 140. The guiding portion 140 is disposed at the base body 110 and has a duct 146. In this embodiment, the duct 146 is, for example, provided in the form of two rib-shaped structures protruding outward relative to the sidewall of the base body 110 and spaced apart from the first position-limiting portion 112a and the second position-limiting portion 112b in the circumferential direction of the base body 110 respectively. The duct 146 extends in the feeding direction F and is in communication with the support channel 116 to not only guide the syringe support 200 being mounted in place along the feeding direction F but also enhance the stability of the syringe support 200 and the syringe base 100 in the circumferential direction upon completion of the assembly process.

Like the position-limiting component slot 117, the abutting component 120 further has a guiding component slot 128. In this embodiment, the guiding component slot 128 is, for example, in the number of two and disposed outside the base opening 126 in the radial direction, whereas the duct 146 extends in the feeding direction F and corresponds in position to a portion of the guiding component slot 128. In some feasible embodiments, the guiding component slot 128 tapers off (i.e., has its width decreasing in the radial direction) toward the duct 146 in the circumferential direction. Thus, the portion of the guiding component slot 128, corresponding in position to the duct 146, is of a smaller width than another portion of the guiding component slot 128, not corresponding in position to the duct 146, in the radial direction. Therefore, when the syringe support 200 is assembled with the syringe base 100 and rotates relative to the syringe base 100, it is easy for the guiding structure of the syringe support 200 to be rotated into the duct 146 to slide along the duct 146.

Preferably, the base body 110 further has an engaging hole 118. The engaging hole 118 is, for example, in the number of two, and the two engaging holes 118 penetrate a portion of the guiding portion 140 respectively such that the guiding portion 140 and the duct 146 are severed by the engaging holes 118 in the feeding direction F. Thus, the syringe support 200 can keep the resilience required for engaging with the syringe base 100 even though the syringe 2 has not been in use for a long time, as described later.

The syringe base 100 further includes a gripping portion 122. The gripping portion 122 is, for example, a bump that extends in the feeding direction F, lies at the abutting component 120 and adjoins the base opening 126 to further fix the syringe support in place in the radial direction and thus preclude unanticipated vibration.

In this embodiment, the first engaging component 130a and the second engaging component 130b include two engaging component sliding connection portions 132 and two engaging arms 134 respectively. The engaging component sliding connection portions 132 are connected to the abutting component 120 through the engaging arms 134 respectively. For the sake of illustration, the engaging component sliding connection portions 132 of the first engaging component 130a in FIG. 4 are, in the top to bottom order, hereinafter referred to as the first engaging component sliding connection portions 132a and the second engaging component sliding connection portions 132b. Likewise, the engaging component sliding connection portions 132 of the second engaging component 130b in FIG. 4 are, in the top to bottom order, hereinafter referred to as the third engaging component sliding connection portions 132c and the fourth engaging component sliding connection portions 132d. The first engaging component sliding connection portions 132a and the second engaging component sliding connection portions 132b are connected to the abutting component 120 through a first engaging arm 134a and a second engaging arm 134b respectively. As shown in FIG. 3 and FIG. 4, the distance between each of the engaging component sliding connection portions 132 and a central axis A of the support channel 116 in the radial direction of the base body 110 gradually changes in the feeding direction F. Specifically speaking, the distance between each of the engaging component sliding connection portions 132 and the central axis A gradually decreases and then gradually increases, in the feeding direction F, to render it easier for the syringe support 200 and the syringe 2 to be inserted into or separated from the syringe base 100 in the feeding direction F or in the direction opposite to the feeding direction F.

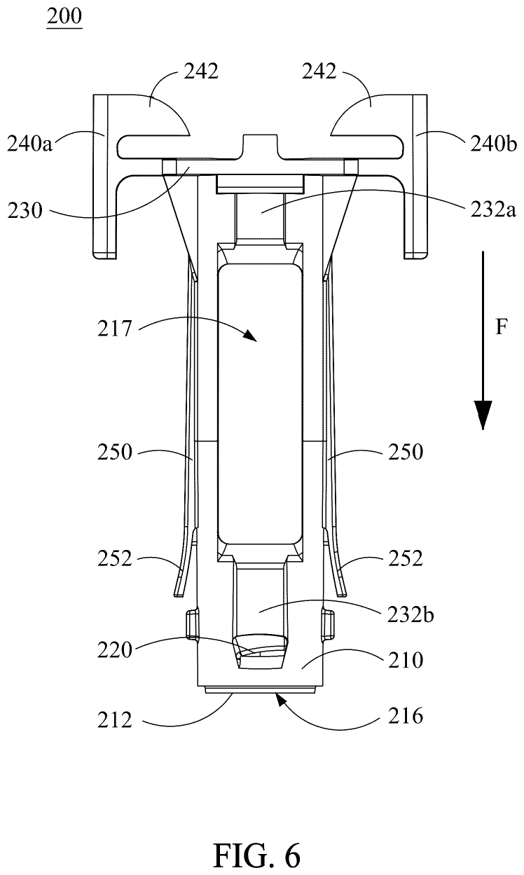

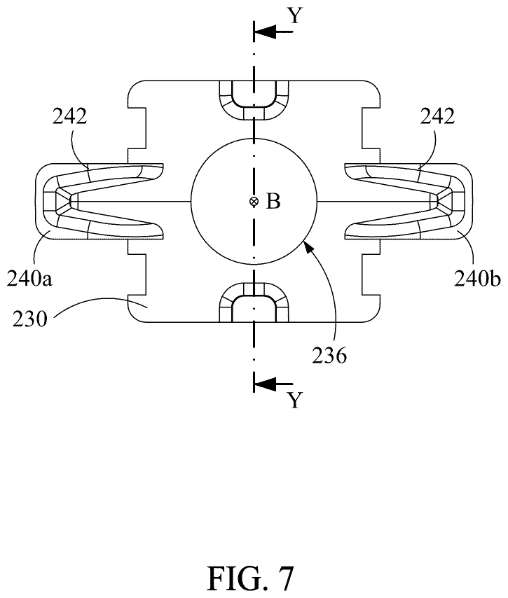

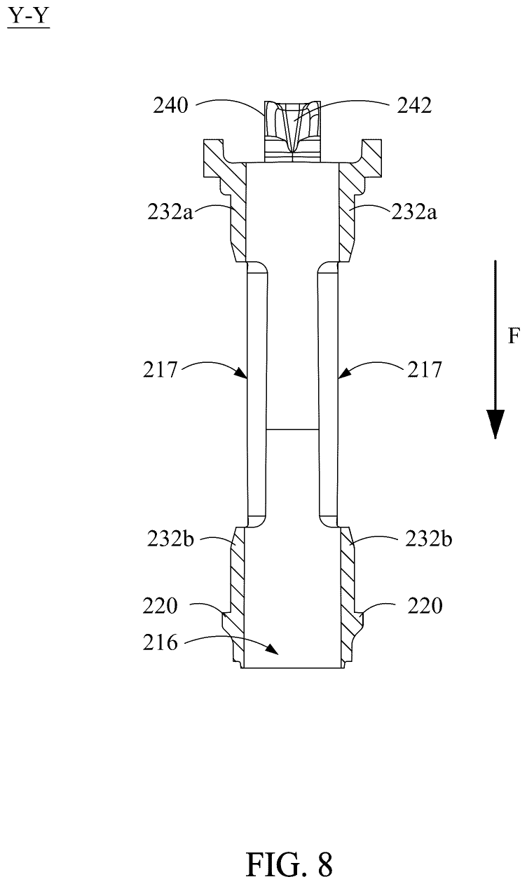

Refer to FIG. 6 through FIG. 8. FIG. 6 is a front view of the syringe support 200 of FIG. 1. FIG. 7 is a top view of the syringe support 200 of FIG. 1. FIG. 8 is a cross-sectional view taken along line Y-Y of FIG. 7. The syringe support 200 in an embodiment is also a substantially hollow tubular structure and includes a support body 210, a position-limiting component 220, a carrying component 230 and at least one wing component 240. The support body 210 has a syringe channel 216. The syringe channel 216 extends in the feeding direction F and penetrates the support body 210. The position-limiting component 220 is disposed at the support body 210 and protrudes relative to the sidewall of the support body 210. In an embodiment, the position-limiting component 220 is, for example, provided in the form of two bumps that protrude outward in a radial direction of the support body 210 and are equally spaced apart in the circumferential direction of the support body 210. The carrying component 230 is disposed at one end of the support body 210 in the feeding direction F and has a support opening 236. The support opening 236 is in communication with the syringe channel 216. The position-limiting component 220 and the carrying component 230 are disposed at the front end and the rear end of the support body 210 in the feeding direction F respectively. The wing component 240 is disposed at the carrying component 230 and protrudes in the feeding direction F and relative to the carrying component 230 in a radial direction of the support body 210. In an embodiment, there is a plurality of wing components 240, for example two. The two wing components 240 are equally spaced apart in the circumferential direction of the support body 210. For the sake of illustration, the wing component 240 shown on the left in FIG. 6 is hereinafter referred to as the first wing component 240a, and the wing component 240 shown on the right in FIG. 6 is hereinafter referred to as the second wing component 240b. The first wing component 240a and the second wing component 240b are adapted to bend sideward or rotate pivotally relative to the carrying component 230.

In an embodiment, the syringe support 200 further includes a guiding component 250. For example, the guiding component 250 is disposed at the support body 210 and is a rib-shaped structure protruding relative to the sidewall of the support body 210. The guiding component 250 is a plurality of guiding components, for example, two. The two guiding components 250 are each spaced apart from the position-limiting component 220 in the circumferential direction of the support body 210. In some embodiments, the guiding component 250 has an end portion 252. The end portion 252 is disposed at a free end of the guiding component 250 and separated from the sidewall of the support body 210. The free end of the guiding component 250 is positioned proximate to the position-limiting component 220. Therefore, the end portion 252 of the guiding component 250 and the engaging hole 118 of the syringe base 100 engage with each other, allowing the syringe base 100 and the syringe support 200 to limit each other in terms of their positions.

As shown in FIG. 6 and FIG. 8, the support body 210 further has a syringe observation slot 217. In an embodiment, the syringe observation slot 217 is a plurality of syringe observation slots, for example, two, and the two syringe observation slots 217 extend in the feeding direction F and penetrate the sidewall of the support body 210 respectively to enable users to conveniently observe the position of the body portion 22, labels affixed to the body portion 22, and the state of the contents of the syringe 2. Preferably, the support body 210 further includes a resilient component abutting portion 212. For example, the resilient component abutting portion 212 is a raised ring-shaped structure disposed at the front end of the support body 210 in the feeding direction F and adapted to abut against the resilient component 300 or allow the resilient component 300 to be assembled around it.

In some embodiments, the syringe support 200 further includes two fastening components. The two fastening components are disposed at the front end and the rear end of the support body 210 in the feeding direction F respectively. For the sake of illustration, the fastening components disposed at the front end and the rear end of the support body 210 in the feeding direction F in FIG. 6 are hereinafter referred to the first fastening components 232a and the second fastening components 232b respectively, and the distance between a surface of each of the fastening components in a radial direction and a central axis B of the syringe channel 236 in a radial direction equals the inner diameter of the syringe channel 236 substantially. Therefore, when the syringe support 200 is located at a using-engaging position and a used-engaging position relative to the syringe base 100 respectively, the first fastening components 232a and the second fastening components 232b each engage with the gripping portion 122 to thereby prevent the syringe support 200 from shaking unnecessarily in the radial direction.

As shown in FIG. 6, the first wing component 240a and the second wing component 240b each include a wing component sliding connection portion 242. The distance between each of the wing component sliding connection portions 242 and a central axis B of the syringe channel 216 in the radial direction of the support body 210 gradually changes in the feeding direction F. In an embodiment, the distance between each of the wing component sliding connection portions 242 and the central axis B gradually decreases and then greatly increases, in the radial direction, to form an engagement-oriented gripping space, for example. Therefore, the syringe 2 can be fixed in place by clamping the bottom of a container of the syringe 2 and positioning it below the first wing component 240a and the second wing component 240b.

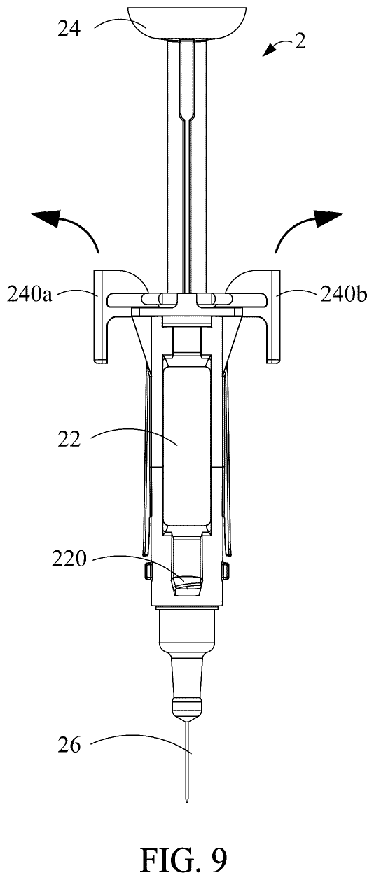

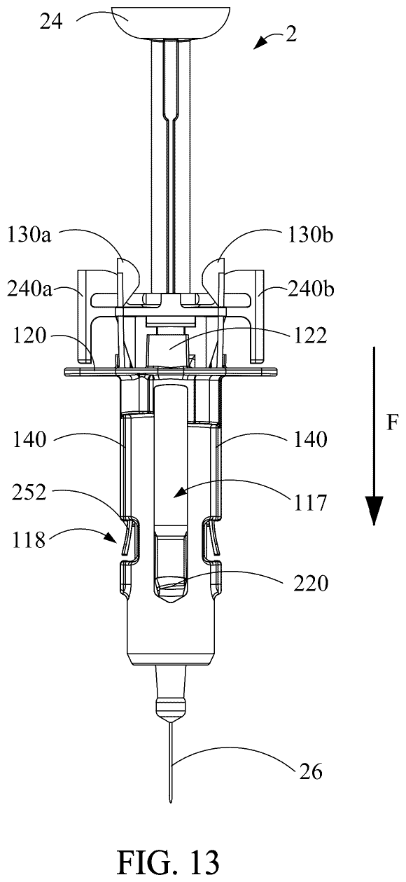

The joint operation of the syringe assembly 1 and the syringe 2 in an embodiment is described in detail below. Referring to FIG. 9 through FIG. 15 consecutively, users can mount the syringe 2 on the syringe assembly 1 by inserting the body portion 22 carrying the needle portion 26 into the syringe channel 216 of the syringe support 200. Since both the first wing component 240a and the second wing component 240b can bend sideward or rotate pivotally relative to the carrying component 230, the two wing components 240 can be shifted outward by a distance (hereinafter referred to the “gap”) in the directions indicated by the bold arrows in FIG. 9 respectively. Since the distance between the circumference of the body portion 22 and the central axis B in the radial direction is greater than the distance between the central axis B and each of the first wing component 240a and the second wing component 240b in the radial direction, the passage of the body portion 22 and the needle portion 26 through the gap is followed by the first wing component 240a and second wing component 240b rebounding resiliently or rotating pivotally to return to their initial positions so as for the syringe 2 to be fixed to the syringe support 200.

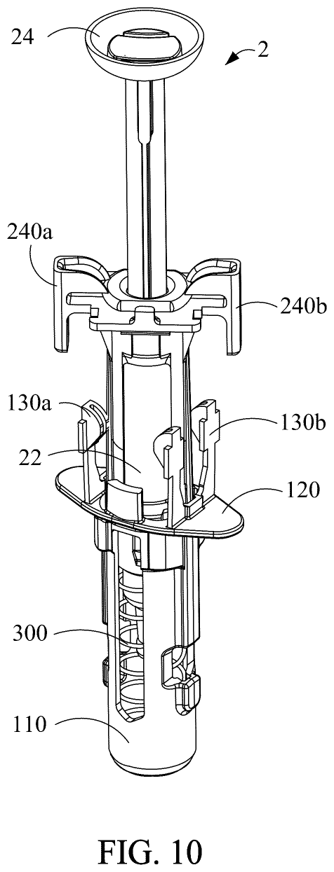

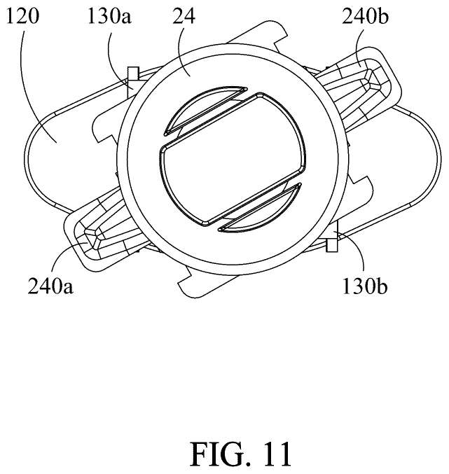

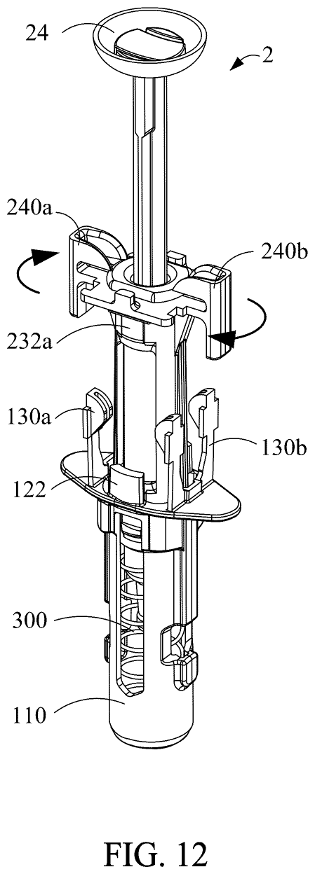

As shown in FIG. 10 and FIG. 11, in an embodiment, the syringe base 100 and the syringe support 200 are adapted to slide and rotate sequentially and then slide sequentially once again so as to undergo the assembly processes. Specifically speaking, users allow a phase difference to be formed between each of the first wing component 240a and the second wing component 240b of the syringe support 200 carrying the syringe 2 and each of the first engaging component 130a and the second engaging component 130b of the syringe base 100 in the circumferential direction to enable the position-limiting component 220 of the syringe support 200 to correspond in position to the position-limiting component slot 127 of the syringe base 100 in the circumferential direction and enable the guiding component 250 to correspond in position to the guiding component slot 128 of the syringe base 100. After that, the syringe support 200 is slightly inserted into the support channel 116 of the syringe base 100 until the bottom of the guiding component 250 or the position-limiting component 220 abuts against the bottom wall of the guiding component slot 128 or the position-limiting component slot 127. Then, the syringe support 200 is rotated by a phase in the directions indicated by the bold arrows in FIG. 12 respectively. Meanwhile, the position-limiting component 220 and the guiding component 250 are guided by the position-limiting component slot 127 and guiding component slot 128 with a gradually-decreasing width into the position-limiting chambers 117 and the duct 146. Then, the syringe support 200 is pushed in the feeding direction F into the syringe base 100 until the first wing component 240a slides across the surface of the first engaging component sliding connection portions 132a and the surface of the second engaging component sliding connection portions 132b and becomes assembled therebetween and until the second wing component 240b slides across the surface of the third engaging component sliding connection portions 132c and the surface of the fourth engaging component sliding connection portions 132d and becomes assembled therebetween. In the course of the slide, the position-limiting components 220 are position-limited by the position-limiting chambers 117 respectively, and the guiding components 250 are guided by the ducts 146 respectively, preventing the syringe support 200 and the syringe base 100 from rotating or deviating relative to each other during the assembly processes but promoting the stability of the assembly and positioning process. The gap between the first engaging component sliding connection portions 132a and the second engaging component sliding connection portions 132b substantially equals the size of the first wing component 240a, and the gap between the third engaging component sliding connection portions 132c and the fourth engaging component sliding connection portions 132d substantially equals the size of the second wing component 240b Thus, upon completion of the assembly processes, the wing components 240 are not only gripped and held between the abutting component 120 and each engaging component sliding connection portion 132 in the feeding direction F but are also limited in position by different engaging component sliding connection portions 132 in the circumferential direction to not only enable the body portion 22 and the needle portion 26 of the syringe 2 and the syringe support 200 to be fixed in place relative to the syringe base 100 but also enable the operating portion 24 to move in the feeding direction F relative to the syringe base 100. Thus, users can directly press downward or pull upward the operating portion 24 to perform injection or withdrawal.

It is noteworthy that the syringe 2, the syringe base 100, and the syringe support 200 may be stored in their assembled states for a long period of time. In an embodiment, the end portion 252 of the guiding component 250 protrudes outward in the radial direction via the engaging hole 118 and remains uncompressed to avoid ending up with resilience fatigue which might otherwise arise from protracted compression by the guiding portion 140. The gripping portion 122 adjoins the base opening 126, and the surface of the first fastening components 232a is substantially in contact with the support channel 116. Thus, after the syringe support 200 and the syringe base 100 have undergone the assembly processes, the gripping portion 122 and the first fastening components 232a interfere with each other and abut against each other to prevent the syringe support 200 from undergoing unnecessary vibration in the radial direction and further enhance assembly stability between the syringe support 200 and the syringe base 100.

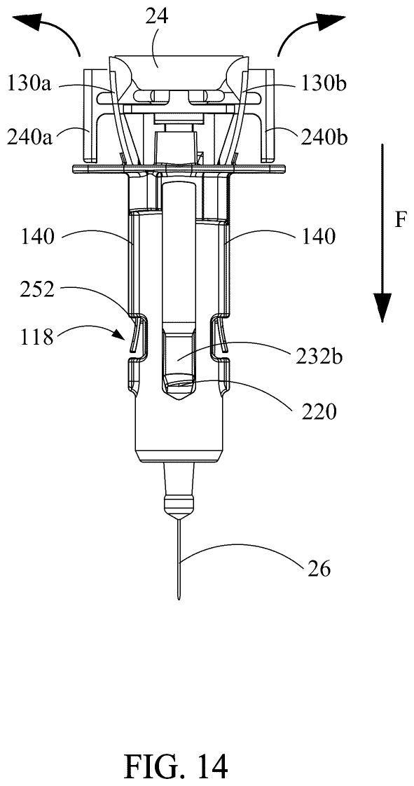

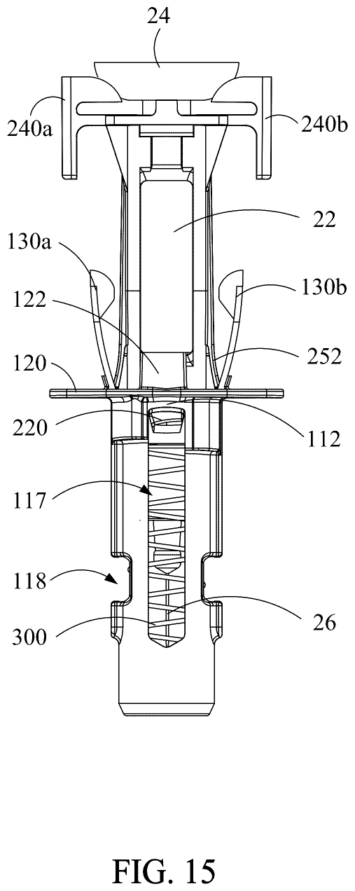

Referring to FIG. 14 and FIG. 15, after users have finished injecting the contents of the body portion 22, the distance between the circumference of the operating portion 24 and the central axis A in the radial direction is greater than the distance between the central axis A and each of the engaging components 130 in the radial direction. Thus, when the operating portion 24 is pushed to reach the vicinity of the body portion 22, both the first engaging component 130a and the second engaging component 130b are squeezed by the operating portion 24 to thereby stretch outward in the directions indicated by the bold arrows in FIG. 14 respectively and undergo plastic deformation. Meanwhile, the first wing component 240a and the second wing component 240b become tightly assembled with the operating portion 24. After that, the operating portion 24, first wing component 240a, and second wing component 240b are pulled and lifted in the direction opposite to the feeding direction F until the position-limiting component 220 abuts against the position-limiting portion 112. At this point in time, the syringe support 200 and the syringe 2 tend to move in the feeding direction F under the resilience force of the resilient component 300, but the end portion 252 of the guiding component 250 which has separated from the duct 146 stretches in the radial direction under its own resilience to thereby abut against the surface of the abutting component 120. Thus, the syringe support 200 is fixed in place relative to the syringe base 100 in the feeding direction F under the resilience force of the resilient component 300 and the abutting relation between the end portion 252 and the abutting component 120. Furthermore, the position-limiting component 220 is engaged with the position-limiting portion 112 and the position-limiting chambers 117 to thereby prevent the syringe support 200 from rotating relative to the syringe base 100 in the circumferential direction so as to ensure that the needle portion 26 can be completely received in the support channel 116 without protruding relative to the syringe base 100. Therefore, ease of use and medical professionals' safety are enhanced.

The gripping portion 122 adjoins the base opening 126, and the surface of the second fastening components 232b is substantially in contact with the support channel 116; thus, when the syringe support 200 and the syringe 2 are pulled upward to reach the position shown in FIG. 15. Then the gripping portion 122 and each second fastening component 232b interfere with each other and abut against each other to prevent the syringe support 200 from undergoing unnecessary vibration in the radial direction and further enhance assembly stability between the syringe support 200 and the syringe base 100. In some embodiments, a stop wall is formed between the junction of the first engaging arm 134a of the first engaging component 130a and the abutting component 120 and the junction of the second engaging arm 134b and the abutting component 120, and a stop wall is also formed between the junction of the abutting component 120 and one of the two engaging arms of the second engaging component 130b and the junction of the abutting component 120 and the other engaging arm of the second engaging component 130b, allowing the end portions 252 in FIG. 15 to abut against the inner sides of the stop walls respectively, or even allowing the end portions 252 to take on hook shapes and thereby engage with the stop walls respectively. Therefore, the release of the end portions 252 from the engaging position is unlikely to occur even under any force exerted by the users, preventing the danger which might otherwise arise from the exposure of the needle portion 26 from the syringe base 100 under a pulling force of the resilient component 300 as a result of the users' careless operation.

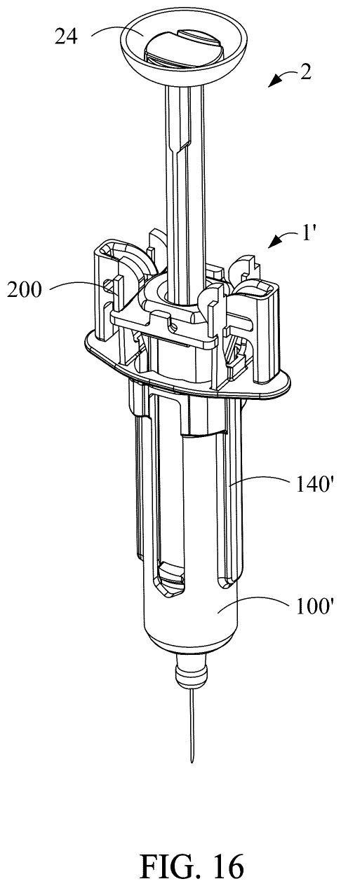

Referring to FIG. 16, there is shown a perspective view of a syringe assembly 1′ disclosed in an embodiment of the present disclosure and assembled with a syringe. The syringe assembly 1′ in an embodiment is distinguished from the syringe assembly 1 in preceding embodiments by a technical feature as the guiding portion 140′ of the syringe base 100′ of the syringe assembly 1′ does not have the engaging hole 118.

Specifically speaking, when the duct 146 is sufficiently deep, the guiding component 250 slides along the duct 146 spontaneously. Therefore, despite the absence of the engaging hole 118, the resilience of the end portion 252 of the guiding component 250 is maintained for a long period of time to facilitate subsequent engagement.

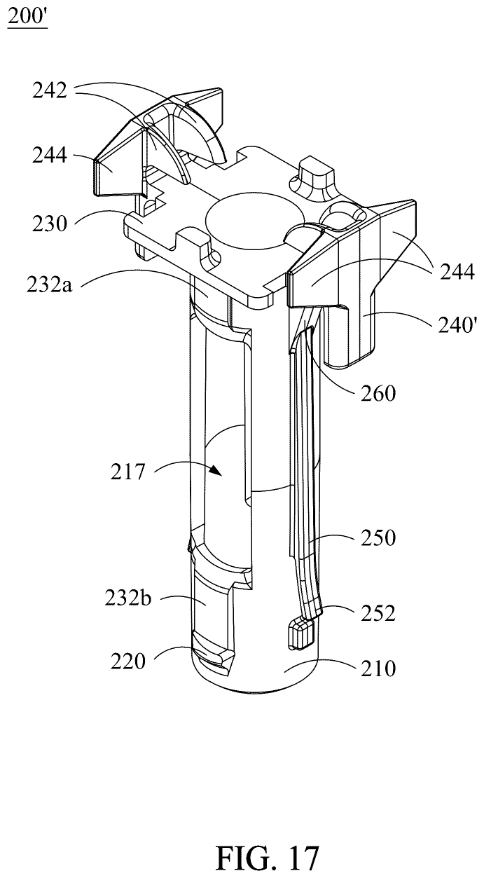

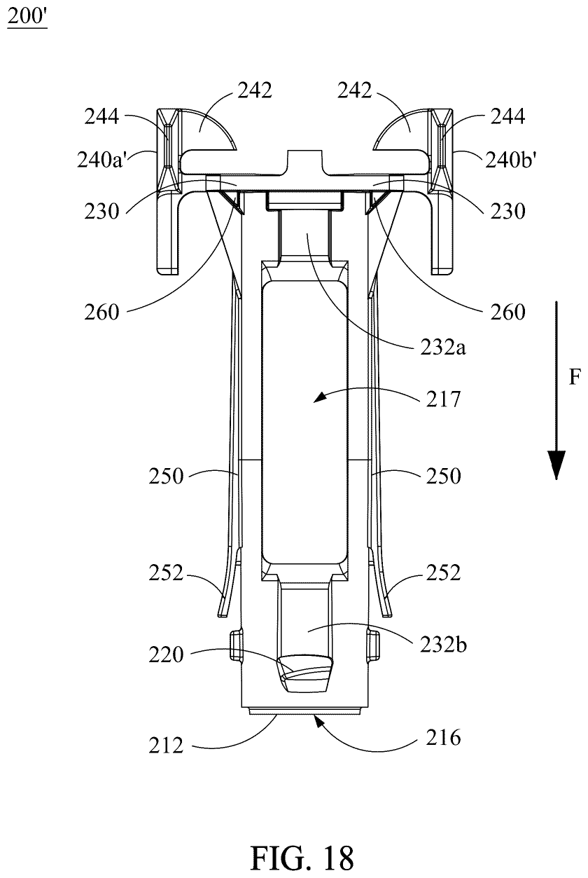

Refer to FIG. 17 and FIG. 18. FIG. 17 is a perspective view of the syringe support of the syringe assembly according to an embodiment of the present disclosure. FIG. 18 is a front view of FIG. 17. The syringe support 200′ in this embodiment is distinguished from the syringe support 200 in the preceding embodiment by technical features as the syringe support 200′ further includes two inclination portions 260, and the wing components 240′ each further include two wing component protection portions 244.

Specifically speaking, after the assembly processes of the syringe base 100

and the syringe support 200 have been finished, the engaging component 130 is located outside the corresponding wing component 240. Thus, when the syringe assembly 1 collides with an external component, the engaging component 130 is likely to be disturbed and thus disengaged from the wing component 240, causing the unanticipated separation of the syringe base 100 and the syringe support 200. In view of this, in an embodiment, the wing components 240′ of the syringe support 200′ each further include two wing component protection portions 244, with the wing component protection portions 244 being, for example, trapezoidal plate-shaped structures and disposed outside the wing component sliding connection portions 242, and the distance between the central axis A and each of the engaging components 130 in the radial direction is less than the distance between the central axis B and each of the wing component protection portions 244 in the radial direction. Therefore, when the syringe support 200 comes into contact with an external component, the wing component protection portions 244 engaging with the engaging component 130 have priority over the engaging component 130 in abutting against or colliding with the external component so as to prevent the engaging component 130 from being disturbed and unexpectedly disengaged.

In an embodiment, the syringe support 200′ further includes two inclination

portions 260, and the inclination portions 260 are, for example, wedge structures disposed between the support body 210, carrying component 230 and wing components 240′. Thus, when the syringe base 100 and the syringe support 200′ are assembled with each other, the syringe support 200′ abuts against the stop walls through the inclination portions 260 and enters the syringe base 100 while being guided by inclination surfaces of the inclination portions 260, enhancing the ease of the assembly processes of the syringe base 100 and the syringe support 200′.

The present disclosure is disclosed above by preferred embodiments. However, persons skilled in the art should understand that the embodiments are illustrative of the disclosure only, but shall not be interpreted as restrictive of the scope of the disclosure. Thus, all variations and replacements equivalent to the embodiments shall be deemed falling within the scope of the disclosure, and technical features of the embodiments can be appropriately combined, replaced, dispensed with and changed on condition that neither conceptual contradictions nor structural conflicts arise. Therefore, the legal protection for the disclosure shall be defined by the appended claims.

What is claimed is:

1. A syringe base, comprising:
a base body comprising a position-limiting portion and having a position-limiting chamber and a support channel, the position-limiting chamber adjoining the position-limiting portion and extending in a feeding direction, the support channel being in communication with the position-limiting chamber and extending in the feeding direction to penetrate the base body;
an abutting component disposed at an end of the base body in the feeding direction and having a base opening and a position-limiting component slot, the base opening being in communication with the support channel, the position-limiting component slot being disposed outside the base opening in a radial direction of the base body and adjoining the position-limiting chamber; and
at least one engaging component disposed at the abutting component, protruding relative to the abutting component, and adapted to bend or rotate relative to the abutting component.

2. The syringe base of claim 1, wherein a width of a portion of the position-limiting component slot positioned distal to the position-limiting chamber in the radial direction is greater than a width of another portion of the position-limiting component slot positioned proximate to the position-limiting chamber in the radial direction.

3. The syringe base of claim 1, further comprising a guiding portion disposed at the base body and having a duct, with the duct extending in the feeding direction and being in communication with the support channel.

4. The syringe base of claim 3, wherein the abutting component further has a guiding component slot disposed outside the base opening in the radial direction, with the duct corresponding in position to a portion of the guiding component slot in the feeding direction.

5. The syringe base of claim 4, wherein a width of the portion of the guiding component slot corresponding in position to the duct in the radial direction is smaller than a width of another portion of the guiding component slot without corresponding in position to the duct in the radial direction.

6. The syringe base of claim 3, wherein the base body further has an engaging hole, and the engaging hole penetrates a portion of the guiding portion.

7. The syringe base of claim 3, wherein the position-limiting portion and the guiding portion are spaced apart in a circumferential direction of the base body.

8. The syringe base of claim 1, further comprising a gripping portion disposed at the abutting component and adjoining the base opening.

9. The syringe base of claim 1, wherein the at least one engaging component is provided in a plurality, with the plurality of engaging components each comprising an engaging component sliding connection portion, and a distance between each of the engaging component sliding connection portions and a central axis of the support channel in the radial direction gradually changes in the feeding direction.

10. The syringe base of claim 1, wherein the at least one engaging component comprises two engaging component sliding connection portions, with the syringe base adapted to be assembled with a syringe support, and a gap between the two engaging component sliding connection portions equals a wing component of the syringe support in size substantially.

11. The syringe base of claim 1, wherein the syringe base is adapted to be assembled with a syringe, with the syringe comprising an operating portion, and a distance between a circumference of the operating portion and a central axis of the support channel in the radial direction is greater than a distance between the at least one engaging component and the central axis in the radial direction.

12. A syringe support, comprising:
a support body having a syringe channel, with the syringe channel extending in a feeding direction and penetrating the support body;
a position-limiting component disposed at the support body and protruding relative to a sidewall of the support body;
a carrying component disposed at the support body and having a support opening, the support opening being in communication with the syringe channel, wherein the position-limiting component and the carrying component are disposed at a front end and a rear end of the support body in the feeding direction respectively;
a guiding component disposed at the support body, protruding relative to the sidewall of the support body, and spaced apart from the position-limiting component in a circumferential direction of the support body; and
at least one wing component disposed at the carrying component, protruding relative to the carrying component, and adapted to bend or rotate relative to the carrying component.

13. The syringe support of claim 12, wherein an end portion of the guiding component and the sidewall of the support body are separate from each other.

14. The syringe support of claim 12, further comprising two fastening components disposed at a front end and a rear end of the support body in the feeding direction respectively, and a distance between a surface of each of the fastening components and a central axis of the syringe channel in a radial direction of the support body substantially equals an inner diameter of the syringe channel.

15. The syringe support of claim 12, wherein the support body comprises a resilient component abutting portion disposed at a front end of the support body in the feeding direction and adapted to abut against a resilient component.

16. The syringe base of claim 12, wherein the support body further has a syringe observation slot extending in the feeding direction and penetrating the sidewall of the support body.

17. The syringe base of claim 12, wherein the at least one wing component is provided in a plurality, with the plurality of wing components each comprising a wing component sliding connection portion, and a distance between each of the wing component sliding connection portions and a central axis of the syringe channel in a radial direction of the support body gradually changes in the feeding direction.

18. The syringe support of claim 12, wherein the at least one wing component is provided in a plurality, and the plurality of wing components are equally spaced apart in the circumferential direction.

19. The syringe support of claim 12, wherein the syringe support is adapted to be assembled with a syringe comprising a body portion, and a distance between a circumference of the body portion and a central axis of the syringe channel in a radial direction of the support base is greater than a distance between the at least one wing component and the central axis in the radial direction.

20. A syringe assembly, adapted to be assembled with a syringe, the syringe assembly comprising:
a syringe base comprising a base body comprising a position-limiting portion and having a position-limiting chamber and a support channel, the position-limiting chamber adjoining the position-limiting portion and extending in a feeding direction, the support channel being in communication with the position-limiting chamber and extending in the feeding direction to penetrate the base body; an abutting component disposed at an end of the base body in the feeding direction and having a base opening and a position-limiting component slot, the base opening being in communication with the support channel, the position-limiting component slot being disposed outside the base opening in a radial direction of the base body and adjoining the position-limiting chamber; and at least one engaging component disposed at the abutting component, protruding relative to the abutting component, and adapted to bend or rotate relative to the abutting component;
a syringe support comprising a support body having a syringe channel, with the syringe channel extending in a feeding direction and penetrating the support body; a position-limiting component disposed at the support body and protruding relative to a sidewall of the support body; a carrying component disposed at the support body and having a support opening, the support opening being in communication with the syringe channel, wherein the position-limiting component and the carrying component are disposed at a front end and a rear end of the support body in the feeding direction respectively; a guiding component disposed at the support body, protruding relative to the sidewall of the support body, and spaced apart from the position-limiting component in a circumferential direction of the support body; and at least one wing component disposed at the carrying component, protruding relative to the carrying component, and adapted to bend or rotate relative to the carrying component, with the syringe support being disposed in the support channel, and the syringe channel adapted to receive the syringe; and
a resilient component disposed between the syringe base and the syringe support.
